(19) United States
(12) Patent Application Publication
(10) Pub. No.: US 2023/0192760 A1
(43) Pub. Date: Jun. 22, 2023

(54) SYSTEMS AND METHODS FOR TYROSINASE-MEDIATED SITE-SPECIFIC PROTEIN CONJUGATION

(71) Applicant: Genentech, Inc., South San Francisco, CA (US)

(72) Inventors: Adel Mahmoud ELSOHLY, South San Francisco, CA (US); Patrick Gwynn Holder, South San Francisco, CA (US)

(21) Appl. No.: 18/052,486

(22) Filed: Nov. 3, 2022

Related U.S. Application Data
(63) Continuation of application No. PCT/US2021/030905, filed on May 5, 2021.
(60) Provisional application No. 63/020,405, filed on May 5, 2020.

(51) Int. Cl. C07K 1/107; C07K 11/02

(52) U.S. Cl. CPC C07K 1/1075; C07K 1/1077; C07K 11/02

(57) ABSTRACT

The present disclosure provides for bioconjugation of biomolecules with functionalized trans-cyclooctenes (TCOs).

### CROSS REFERENCE TO RELATED APPLICATIONS

This application claims priority to U.S. Provisional Application 63/020,405, filed May 5, 2020 and is incorporated herein by reference in its entirety.

### BACKGROUND

Site-specific bioconjugation to native amino acids remains a challenge for chemists. Discerning surface-abundant amino acids like lysine, aspartic acid and glutamic acid from similar neighbors with chemical reagents is usually difficult; except in rare cases, the functional group of an amino acid side chain overwhelms any subtle variations in steric or electronic local environment that might lead to enhanced reaction kinetics at a single site. In contrast, installation of amino acids that are infrequently found on protein surfaces (cysteine in particular) can provide site selectivity, which can be leveraged for site-specific chemical reaction.

A similar specificity is achieved by enzymes, which have evolved to isolate functional groups in a unique steric and electronic context. Many enzymes react with amino acid sidechains. They can be used to install or remove post-translational modifications like phosphates, glycans, or lipids. For bioconjugation, a useful subclass of these enzymes is those evolved for tissue crosslinking, because they are amino acid sequence specific. This property is exploited by protein chemists in the case of transglutaminase (TG), formylglycine-generating enzyme (FGE), sortase, phosphopantethienyl transferase (PPTase), and laccase, among others. Site-specific reaction is achieved through recombinant installation of the amino acid(s) recognized by the enzyme. Tyrosinase is also in this class of enzymes.

Tyrosinase performs a two-step oxidation of tyrosine to dihydroxyphenylalanine (DOPA), and subsequently, the o-quinone of DOPA (dopaquinone). In nature, dopaquinone is a precursor to both eumelanin and, upon combination with cysteine, pheomelanin, the aromatic skin and hair pigment polymers; in the lab, scientists can exploit this reactivity to modify recombinant proteins. Tyrosinase recognizes only the phenol sidechain of tyrosine and it is able to convert it to the o-quinone without requiring specificity from flanking amino acids. Importantly, tyrosine is quite rarely found on protein surfaces; in those few occurrences, its sidechain tends to be occluded by hydrophobic packing. This results in very few tyrosine residues for which the phenol is sufficiently extended to reach into the active site of tyrosinase. As such, it is possible to install tyrosine residues and achieve site-specific protein modification. This method has been put to use in a variety of examples, including conjugation of cytotoxic cargos through Diels-Alder cycloaddition, protein—protein conjugation of Cas9, and profiling the oxidative coupling of anilines.

These advances in conjugation notwithstanding, yields and conjugate product stability continue to be issues in biomolecule conjugation. The present disclosure provides embodiments that meet these and other needs.

### BRIEF SUMMARY OF EMBODIMENTS

In embodiments, there are provided compositions including a cycloadduct of a functionalized trans-cycloöctene (TCO) with an ortho-quinone, wherein the ortho-quinone is present in a biomolecule.

In embodiments, there are provided compositions of formula (I):

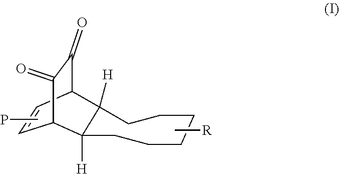

wherein P is a protein or peptide, and R comprises a second protein, a second peptide, a drug, a nucleic acid, an oligosaccharide, a polymer, an oligomer, a dendrimer, a label, or a substrate surface, any of which are optionally attached through a linker.

In embodiments, there are provided methods comprising adding a functionalized trans-cycloöctene (TCO) to an ortho-quinone present in a biomolecule, thereby forming a cycloadduct between the functionalized TCO and the ortho-quinone.

In embodiments, there are provided methods comprising providing a functionalized trans-cycloöctene (TCO), adding a protein or peptide comprising a phenolic moiety to the functionalized TCO, and generating an ortho-quinone from the phenolic moiety, wherein the functionalized TCO is allowed to react with the ortho-quinone to form a cycloadduct.

In embodiments, there are provided antibody conjugates formed by action of tyrosinase on a phenolic residue in the antibody in the presence of a functionalized trans-cycloöctene (TCO), wherein the antibody conjugate is stable for at least one month at 37° C. in phosphate buffered saline.

In embodiments, there are provided protein conjugates formed by action of tyrosinase on a phenolic residue in the protein in the presence of a functionalized trans-cycloöctene (TCO), wherein the protein conjugate is stable for at least one month at 37° C. in phosphate buffered saline.

In embodiments, there are provided mixtures comprising a biomolecule having a phenolic moiety, a tyrosinase, and a functionalized trans-cyclooctene.

### BRIEF DESCRIPTION OF THE DRAWINGS

FIG. 1 shows mass spectra resolved at different times indicating the transient generation of dopaquinone in a human IgG1 Fab engineered to display a C-terminal peptide tag containing a tyrosine residue. The same Fab lacking tyrosine results in no modification.

FIG. 2A-C shows conjugation of dienophiles with dopaquinone in situ: in column FIG. 2A, the top pane shows product formation time-course with dienophile TCO, middle pane dienophile BCN, and bottom pane DBCO, each reagent serving as a trap for Fab containing dopaquinone generated in situ. The same timecourse is shown for transiently generated o-quinone (column FIG. 2B), and the side product Fab-Fab dimer (column Fig. C), which are formed during the progress of the reaction.

FIG. 3 shows deconvoluted LCMS spectra of a typical reaction and purification demonstrating compatability of cycloadduct linkages with standard laboratory procedures. The spectra from top to bottom show: starting material (Fab containing a C-terminal “DRY” peptide tag), the reaction after 1 h of reaction time showing the formation of conjugated product at M+432 Da (corresponding to 14 Da for +O, −H₂ and 428 Da for the TCO-PEG₄-COOH reagent), the reaction after 16 hour of reaction time demonstrating about 91% yield, the purified pool after elution at pH 2.7 from a KappaSelect affinity column, and the final product formulated in PBS.

FIGS. 4A-B. shows stability of Diels-Alder cycloadducts, formed by tyrosinase-mediated bioconjugation, against PBS, pH 7.4, 37° C. over the course of multiple months for three variants of a Fab displaying an engineered C-terminal peptide tag: DRY, DRGY, and GGY. In each case, conjugates consisted of a mass shift corresponding to O-atom addition (16 Da), loss of H₂ (−2 Da), and addition of the mass of the TCO dienophile (TCO-PEG₄-carboxylic acid, 417.5); calculated total mass shift 431.5 Da. For Fab-GGY, found M+431.5; for Fab-DRY, found M+431.6; for Fab-DRGY, found 431.7. Each Fab-TCO-PEG₄-COOH conjugate was formulated at 5 mg/mL in PBS and sterilized in a tissue culture hood by passing through a 0.22 μm syringe filter. The container was sealed under ambient conditions, and then stored at 37° C. At each specified timepoint, an aliquot of sample was removed from the container and analyzed by LCMS for deconjugation. The amount of conjugate remaining was calculated as a percent of deconvoluted mass peak abundances. The left pane (FIG. 4A) shows the LCMS spectra recorded for the Fab-DRY protein. Peak abundance at the conjugate MW and the Fab starting material MW were largely unchanged, but there was an about 1% increase in abundance of a Fab fragment corresponding to loss of the C-terminal Tyr residue (calc'd M−163.5, found M−163.6). The right pane (FIG. 4B) is a summary of the amount of conjugate remaining at each timepoint for the three engineered, conjugated Fabs.

FIG. 5 shows a timecourse of reagent generation based on FIG. 4: proximal Arg leads to faster initial rate (1 hour) and higher overall yield. This provides an increase from 77% to 92.5% conjugation efficiency.

### DETAILED DESCRIPTION

### I. GENERAL

The present disclosure relates to bioconjugation reactions that provide site-specific modification of proteins through enzymatic generation of reactive ortho-quinone intermediates from either tyrosine residues or similar phenolic moieties. As disclosed herein, the enzymatically generated o-quinone rapidly reacts with dienophiles, such as cyclooctynes and cyclooctenes. While the yields of conjugation adducts and stability of conjugates at physiological pH and temperature can vary, the present embodiments have found a useful reaction partner for the transient ortho-quinone that provides a conjugated product in good yields and is stable under physiological conditions for prolonged periods.

### II. DEFINITIONS

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Alkylene” refers to a straight or branched, saturated, aliphatic radical having the number of carbon atoms indicated (i.e., C₁₋₆ means one to six carbons), and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative C₁₋₄ alkenylene groups include, but are not limited to, methylene, ethylene, propylene, isopropylene, butylene, isobutylene, and sec-butylene.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Alkoxy-alkyl” refers to a radical having an alkyl component and an alkoxy component, where the alkyl component links the alkoxy component to the point of attachment. The alkyl component is as defined above, except that the alkyl component is at least divalent, an alkylene, to link to the alkoxy component and to the point of attachment. The alkyl component can include any number of carbons, such as C₁₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The alkoxy component is as defined above. Examples of the alkyl-alkoxy group include, but are not limited to, 2-ethoxy-ethyl and methoxymethyl.

“Hydroxyalkyl” or “alkylhydroxy” refers to an alkyl group, as defined above, where at least one of the hydrogen atoms is replaced with a hydroxy group. As for the alkyl group, hydroxyalkyl or alkylhydroxy groups can have any suitable number of carbon atoms, such as C₁₋₆. Exemplary C₁₋₄ hydroxyalkyl groups include, but are not limited to, hydroxymethyl, hydroxyethyl (where the hydroxy is in the 1- or 2-position), hydroxypropyl (where the hydroxy is in the 1-, 2- or 3-position), hydroxybutyl (where the hydroxy is in the 1-, 2-, 3- or 4-position), 1,2-dihydroxyethyl, and the like.

“Halogen” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Amino” refers to an —N(R)₂ group where the R groups can be hydrogen, alkyl, alkenyl, alkynyl, cycloalkyl, heterocycloalkyl, aryl, or heteroaryl, among others. The R groups can be the same or different. The amino groups can be primary (each R is hydrogen), secondary (one R is hydrogen) or tertiary (each R is other than hydrogen).

“Alkylamine” refers to an alkyl group as defined within, having one or more amino groups. The amino groups can be primary, secondary or tertiary. The alkyl amine can be further substituted with a hydroxy group to form an amino-hydroxy group. Alkyl amines useful in the present invention include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group. One of skill in the art will appreciate that other alkyl amines are useful in the present invention.

“Heteroalkyl” refers to an alkyl group of any suitable length and having from 1 to 3 heteroatoms such as N, O and S. Heteroalkyl groups have the indicated number of carbon atoms where at least one non-terminal carbon is replaced with a heteroatom. Additional heteroatoms can also be useful, including, but not limited to, B, Al, Si and P. The heteroatoms can also be oxidized, such as, but not limited to, —S(O)— and —S(O)₂—. For example, heteroalkyl can include ethers, thioethers and alkyl-amines. Heteroalkyl groups do not include peroxides (—O—O—) or other consecutively linked heteroatoms. The heteroatom portion of the heteroalkyl can replace a hydrogen of the alkyl group to form a hydroxy, thio or amino group. Alternatively, the heteroartom portion can be the connecting atom, or be inserted between two carbon atoms.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

“Alkyl-cycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

“Heterocycloalkyl” refers to a saturated ring system having from 3 to 12 ring members and from 1 to 5 heteroatoms of N, O and S. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, 4, or 5, or 1 to 2, 1 to 3, 1 to 4, 1 to 5, 2 to 3, 2 to 4, 2 to 5, 3 to 4 or 3 to 5. The heterocycloalkyl group can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. The heterocycloalkyl group can include groups such as aziridine, azetidine, pyrrolidine, piperidine, azepane, diazepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline, diazabicycloheptane, diazabicyclooctane, diazaspirooctane or diazaspirononane. Heterocycloalkyl groups can be unsubstituted or substituted. For example, heterocycloalkyl groups can be substituted with C₁₋₆ alkyl or oxo (═O), among many others. Heterocycloalkyl groups can also include a double bond or a triple bond, such as, but not limited to dihydropyridine or 1,2,3,6-tetrahydropyridine.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxzoalidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. The heteroatoms can also be oxidized, such as, but not limited to, —S(O)— and —S(O)₂—. Heteroaryl groups can include any number of ring atoms, such as, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “amino acid” as used herein refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

As used herein, the term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

As used herein, the term “antibody fragment” refers to a portion of an antibody. Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen (see, e.g., FUNDAMENTAL IMMUNOLOGY (Paul ed., 4th ed. 2001). As appreciated by one of skill in the art, various antibody fragments can be obtained by a variety of methods, for example, digestion of an intact antibody with an enzyme, such as pepsin; or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries (see, e.g., McCafferty et al., (1990) Nature 348:552). The term “antibody” also includes bivalent or bispecific molecules, diabodies, triabodies, and tetrabodies. Bivalent and bispecific molecules are described in, e.g., Kostelny et al. (1992) J. Immunol. 148:1547, Pack and Pluckthun (1992) Biochemistry 31:1579, Hollinger et al.(1993), PNAS. USA 90:6444, Gruber et al. (1994) J Immunol. 152:5368, Zhu et al. (1997) Protein Sci. 6:781, Hu et al. (1996) Cancer Res. 56:3055, Adams et al. (1993) Cancer Res. 53:4026, and McCartney, et al. (1995) Protein Eng. 8:301.

As used herein, the term “cycloadduct” refers to the product of the [4+2] Diels-Alder reaction between the disclosed transient o-quinones and functionalized TCO dienophile traps.

As used herein, the term “functionalized trans-cycloöctene” or “functionalized TCO” refers to any TCO bearing a functional moiety that can include an organic functional group handle, a linker, an organic compound of interest, and combinations of these. Functionalized TCOs include those comprising PEG linkers, though any linker may be employed. Functionalized TCOs may be commercially available (Broadpharm, Click Chemistry Tools, Jena Bioscience, for example) or prepared by synthesis.

As used herein, the term “biomolecule” refers to any of amino acids, proteins, peptides, oligosaccharides, monosaccharides, amino acids, nucleic acids, including RNA and DNA.

As used herein, the term “modified DNA or RNA” refers to any nucleic acid comprising a modification to incorporate a phenolic residue thereby allowing reaction with tyrosinase.

As used herein, the term “linker” refers to any organic fragment that connects the TCOs disclosed herein to a compound of interest for coupling with tyrosinase generated ortho-quinones, as disclosed herein. In embodiments, the linker is hydrophilic, although it is not so limited. Linkers can include alkyl chains with one or more carbon atoms substituted with heteroatoms, such as O, N, or S. Linkers can include any of the organic functional groups defined herein above.

As used herein, the term “nucleic acid” refers to single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g, H⁺, NH₄⁺, trialkylammonium, tetraalkylammonium, Mg²⁺, Na⁺ and the like. A nucleic acid includes polynucleotide and oligonucleotide. A nucleic acid may be composed entirely of deoxyribonucleotides, entirely of ribonucleotides, or chimeric mixtures thereof. The nucleotide monomer units may include naturally occurring nucleotides and nucleotides analogs. Nucleic acids may range in size from a few monomeric units, e.g, 5-40, to several thousands of monomeric nucleotide units. Nucleic acids include, but are not limited to, genomic DNA, cDNA, hnRNA, mRNA, rRNA, tRNA, RNAi, anti-sense nucleic acids, fragmented nucleic acid, nucleic acid obtained from sub-cellular organelles such as mitochondria or chloroplasts, and nucleic acid obtained from microorganisms or DNA or RNA viruses that may be present on or in a biological sample.

### III. CONJUGATES

In embodiments, there are provided compositions comprising a cycloadduct of a functionalized trans-cycloöctene (TCO) with an ortho-quinone, wherein the ortho-quinone is present in a biomolecule. In embodiments, a generic reaction is provided by the following chemical equation (I):

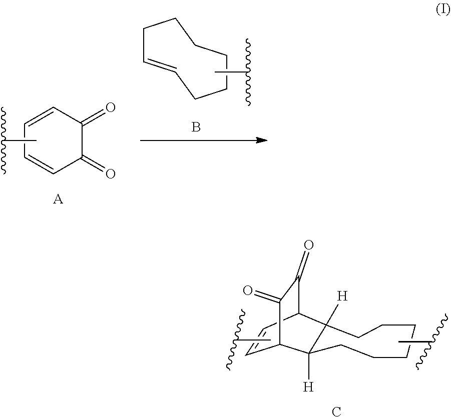

In equation (I), A is an ortho-quinone (o-quinone) within the framework of any biomolecule, as described herein furtherbelow. Transient intermediate A reacts with functionalized TCO B to form cycloadduct C. In embodiments, intermediate A is formed by action of a tyrosinase enzyme on a phenol-containing residue within the biomolecule. In embodiments, the phenol containing residue can be the amino acid tyrosine. In embodiments, the phenol containing residue can be a catechol. In embodiments, transient o-quinone A can also be generated by chemical reaction in lieu of enzymatic oxidation. Such chemical reactions include, without limitation, oxidation with iodoxybenzoic acid, silver oxide, Fremy's salt, K₃Fe(CN)₆, sodium periodate and the like. As indicated in chemical equation (I), the reaction chemistry is intended to be generally applicable coupling any two components through this [4+2] cycloaddition, i.e., Diels Alder reaction.

In embodiments, the biomolecule can include a lipid, a carbohydrate, a nucleic acid, a protein, or any other biological material.

In embodiments, the biomolecule is a lipid, which can include, for example, phospholipids, sphingolipids, glycerolipids, such as triglycerides, free fatty acids, fatty alcohols, sterols, and the like. While lipids vary in structure, they generally all comprise sufficient functional group handles for attachment of a 4-hydroxybenzyl ether group for the purpose of generating a coupling partner from which the o-quinone can be generated. Where attachment of the requisite phenolic precursor as at the polar head of a lipid, a linker may be employed between the lipid and the phenol. This linker may have the same general chemical character as the polar head (i.e., be generally hydrophilic). Such linkers may include small polyethylene glycol units, for example.

In embodiments, the biomolecule is a carbohydrate, for example, a sugar, a starch or a cellulose material. In embodiments, the carbohydrate, can be for example, a monosaccharide or an oligosaccharide. In embodiments, such sugar-based substrates may terminate as phenol-containing glycosides. By way of example, such oligosaccharides can be represented by the following general Formula A:

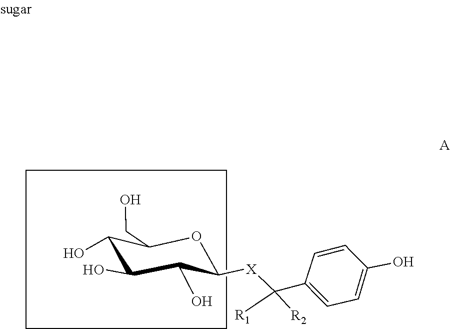

wherein “sugar” is any monosaccharide or oligosaccharide (drawn here generically with a glucose unit, but intended to mean any mono- or oligosaccharide); X is O or NH; and R₁ and R₂ are independently selected from carboxylic acid, ester, alkyl, and hydrogen. In embodiments, R₁ and R₂ are both hydrogen. In embodiments, R₁ is hydrogen and R₂ is carboxylic acid or its ester. In embodiments, the biomolecule is an oligosaccharide, the oligosaccharide has a phenol-containing moiety at its reducing end. In embodiments, monosaccharides or oligosaccharides need not be limited to attachment at the reducing sugar terminus. Accordingly, in embodiments, any 4-hydroxybenzyl ether (or catechol equivalent) at any desired position of a sugar can be targeted for installation of the o-quinone precursor. Exemplary oligosaccharides can include, without limitation, hyaluronic acid, alginate, heparin, and heparain sulfate.

In embodiments, the biomolecule is a nucleic acid, for example, a modified or unmodified DNA or RNA. Such modified structures are configured to incorporate phenolic residues. Incorporation can include using nucleic acids displaying 5′ or 3′ amino groups, as are commercially available from numerous vendors, and functionalizing using aqueous amide-coupling conditions with 3-(4-hydroxyphenyl)propionic acid. In embodiments, modified DNA or RNA can be double- or single-stranded.

In embodiments, the biomolecule is a peptide or protein. For example, the peptide or protein can be an enzyme, a cell surface protein, a cytokine, a chemokine, a protein toxin, or a hormone, as non-limiting examples. In embodiments, the biomolecule can be an antibody or antibody fragment. In embodiments, the antibody can be a monoclonal or polyclonal antibody. In embodiments, the antibody is a monoclonal antibody.

In embodiments, the ortho-quinone can be derived from a phenol-containing moiety. In embodiments, the phenol-containing moiety is tyrosine. In embodiments, the phenol-containing moiety is a catechol. In embodiments, the phenol containing moiety is a 4-hydroxyalkyl phenol residue. In embodiments, the phenol containing moiety may be represented by the following general Formula (B):

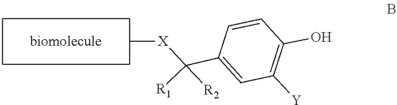

wherein “biomolecule” is as defined herein; X is O or NH; Y is H or OH; and R₁ and R₂ are independently selected from carboxylic acid, ester, alkyl, and hydrogen.

In embodiments, the tyrosine is site-specifically engineered into a protein. In embodiments, tyrosine can be made available (accessible) to reagents in solution through the use of adjacent amino acid residues that promote solubility. Non-limiting examples for this purpose can include, small, flexible and hydrophilic amino acids including glycine, serine, glutamic acid, aspartic acid, and arginine are compatible.

The functionalized TCO can carry any molecule or multiple molecules of interest, where, for example a branched linker is employed. In embodiments, the functionalized TCO comprises a protein, a second peptide, a drug, a nucleic acid, an oligosaccharide, a polymer, an oligomer, a dendrimer, a label, a diagnostic agent, a dual-function therapeutic-diagnostic agent, or a substrate surface, any one or more of which are optionally attached through a linker, wherein the linker is optionally branched.

In embodiments, the functionalized TCO carries more than one molecule of interest. In some such embodiments, the linker is branched and carries two or more of a protein, a peptide, a drug, a nucleic acid, an oligosaccharide, a polymer, an oligomer, a dendrimer, a label, a diagnostic agent, a dual-function therapeutic-diagnostic agent, or a substrate surface. In embodiments, the branched linker may comprise a PABA-releasing moiety.

In embodiments, the functionalized TCOIn embodiments, the nucleic acid is a RNAi or an anti-sense oligonucleotide.

In embodiments, the label is a fluorophore, a radiolabel, a chemiluminescent label, a DNA barcode, a RNA barcode, or a peptide tag.

In embodiments, the substrate surface is a polymer bead, a well bottom of a well-plate, or a polymer slide surface.

In embodiments, there are provided compositions of formula (I):

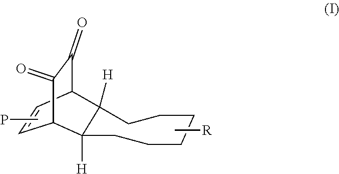

wherein P is a protein or peptide; and

R comprises a second protein, a second peptide, a drug, a nucleic acid, an oligosaccharide, a polymer, an oligomer, a dendrimer, a label, or a substrate surface, any of which are optionally attached through a linker.

In embodiments, P is an antibody or antibody fragment. In embodiments, P is a therapeutic antibody. In embodiments, the antibody or antibody fragment can be trastuzumab, brentuximab, enfortumab, gemtuzumab, inotuzumab, polatuzumab, as non-limiting examples. Others include abciximab, ranibizumab, certolizumab, adalimumab, alefacept, alemtuzumab, basiliximab, belimumab, bezlotoxumab, canakinumab, certolizumab pegol, cetuximab, daclizumab, denosumab, efalizumab, golimumab, inflectra, ipilimumab, ixekizumab, natalizumab, nivolumab, olaratumab, omalizumab, palivzumab, panitumumab, pembrolizumab, rituximab, tocilizumab, secukinumab, and ustekinymab. Still other include aducanumab, teplizumab, dostarlimab, tanezumab, margetuximab, naxitamab, belantamab, oportuzumab monatox, REGNEB3, narsoplimab, tafasitamab, satralizumab, inebilizumab, leronlimab, sacituzumab, teprotumumab, isatuximab, eptinezumab, enfortumab, crizanlizumab, brolucizumab, risankizumab, romosozumab, caplacizumab, ravulizumab, emapalumab, cemiplimab, fremanezumab, moxetumomab, galcanezumab, lanadelumab, mogamuizumab, erenumab, tildrakizumab, ibalizumab, burosumab, duravalumab, emicizumab, benralizumab, ocrelizumab, guselkumab, inotuzumab, sarilumab, dupilumab, avelumab, brodalumab, atezolizumab, bezlotoxumab, olaratumab, reslizumab, obiltoxaximab, ixekizumab, daratumumab, elotuzumab, necitumumab, idarucizumab, alirocumab, mepolizumab, evolocumab, dinutuximab, nivolumab, blinatumomab, pembrolizumab, ramucirumab, vedolizumab, siltuximab, obinutuzumab, raxibacumab, pertuzumab, adotrastuzumab, brentuximab, and belimumab.

In the present embodiments, antibodies or antibody fragments can be used to carry a drug payload, wherein the drug payload is conjugated by the methods disclosed herein. In embodiments, antibodies or antibody fragments can be coupled with labels that facilitate detection, such as fluorescent labels, radiolabels, chemiluminescent labels, DNA barcodes, RNA barcodes, peptide tags, and the like.

In embodiments, P is an enzyme. In embodiments, P is a cell surface protein. In embodiments, the P is a cytokine. In embodiments, P is a chemokine. In embodiments, P is a protein toxin. In embodiments, P is a hormone.

In embodiments, R can include a variety of different molecules, moieties and compounds. For example, antibodies, antibody fragments, targeting molecules, therapeutic agents, cancer chemotherapeutics, immunotherapeutics, labels, sugars, polymers, polymer bead surfaces, sensor surfaces and the like.

In embodiments, R can include a targeting molecule. Targeting molecules can include any small molecule ligand for any biological receptor, including cell surface receptors. Targeting molecules can include, for example, RNA, DNA, and peptides.

In embodiments, R comprises a therapeutic agent. Such agents include, without limitation, chemotherapeutic agents, immunotherapeutic agents, such as immune agonists, cytokines, and chemokines, and any mixtures of such agents. Chemotherpeutic agents can include anti-cancer agents. Therapeutic agents also include antibodies, antibody fragments, fusion proteins, and the like.

In embodiments, R may comprise a label that allows for detection. In embodiments, R comprises a radiolabel. In embodiments, R comprises a fluorescent label. In embodiments, R comprises a phosphorescent label. In embodiments, R comprises a dye.

In embodiments, R can comprise any surface to which attachment of a biomolecule is desired. In embodiments, R may comprise a polymer bead. In embodiments, R may comprise a silicon surface or a coated silicon surface. In embodiments, R may comprise a glass surface. In embodiments, R may comprise a sensor surface. Surface chemistries are well-known in the art and include the use of aminosilanes, for example, as functional group handles. Commercial TCOs are readily available with appropriate functional group handles, including alcohols, carboxylic acids, amines and the like, to integrate with conventional surface functionalization chemistries.

In embodiments, R comprises a linker. In embodiments, the linker may comprise a polyethylene glycol (PEG) unit. In embodiments, the number of PEG units may vary from 1 to about 50, or about 1 to 20, or about 1 to 10. Longer PEG groups are also available as needed and depending on the nature of the actual coupling partners P and R being coupled in the bioconjugation. In embodiments, the linker may be any hydrophilic linking group. In embodiment, the linker may comprise a polymer. In embodiments, the linker may comprise a peptide. In principle, the linker can be any group that allows connectivity between a desired target of immobilization and TCO. Factors in selecting an appropriate linker may include, without limitation, steric requirements, water solubility, and hydrophilicity.

In embodiments, bioconjugated compositions are provided having structures of formula (II):

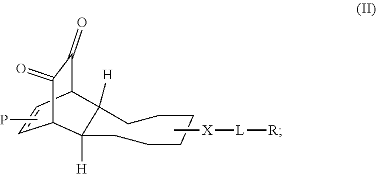

wherein L is a linker or bond and X is O, S, or NH, and P and R are defined as above.

In embodiments, bioconjugated composition are provided having structures of formula (III):

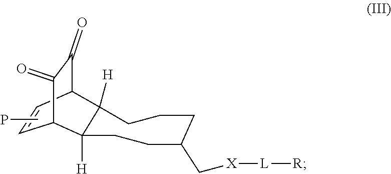

wherein L is a linker or bond and X is O, S, or NH, and P and R are defined as above.

Linkers L may include any arrangement of organic groups connecting a desired organic moiety to TCO. In general, a linker may comprise from 1 to 20 carbon atoms, any of which can replaced with a heteroatom, such as O, NH, or S. In embodiments, any organic functional group can be incorporated in the linker, including the organic functional group defined hereinabove. Non-limiting examples include carbamates, amides, oxo, ureas, and the like. In embodiments, linkers may be branched, once, twice, or any desired number of times to increase the valency of what is attached via the TCO fragment. For example, a branched linker may be employed to attach multiple copies of an oligosaccharide or drug. In embodiments, branched linkers may be used to deliver two, three, or four different organic moiety via the TCO coupling partner.

In embodiments, there are provided antibody conjugates formed by action of tyrosinase on a phenolic residue in the antibody in the presence of a functionalized trans-cycloöctene (TCO), wherein the antibody conjugate is stable for at least one month at 37° C. in phosphate buffered saline.

In embodiments, the antibody comprises a phenolic residue that is tyrosine. Incorporation of tyrosine into the antibody can be accomplished by any method, including, for example, site selective engineering, native chemical ligation, sortase-mediated peptide ligation, transglutaminase-mediated peptide bioconjugation, in vitro translation, in vivo translation, etc. See, e.g., U.S. Pat. Nos. 8,030,074; 8,980,581; and 9,102,932; each of which is incorporated by reference in its entirety, including for all methods, reagents, compositions, and teachings therein.

In embodiments, antibodies can be coupled to functionalized TCOs comprising a label a fluorescent label, a radiolabel or the like.

In embodiments, antibodies can be coupled to functionalized TCOs comprising a drug or other therapeutic agent, including chemotherapeutics, immunotherapeutics and the combinations thereof.

In embodiments, there are provided protein conjugates formed by action of tyrosinase on a phenolic residue in the protein in the presence of a functionalized trans-cycloöctene (TCO), wherein the protein conjugate is stable for at least one month at 37° C. in aqueous solution. In embodiments, the aqueous solution is phosphate buffered saline.

In embodiments, the protein comprises a phenolic residue that is tyrosine.

In embodiments, the protein is coupled to a functionalized TCO comprising a fluorescent label, radiolabel, or similar moiety conferring detection of the protein.

In embodiments, the protein is coupled to a functionalized TCO comprising a drug or other therapeutic agent, including chemotherapeutics, immunotherapeutics and the combinations thereof.

In embodiments, the protein is coupled to a functionalized TCO comprising an oligosaccharide, giving access to glycoprotein structures.

In embodiments, there are provided mixtures comprising a biomolecule having a phenolic moiety, a tyrosinase, and a functionalized trans-cyclooctene. In some embodiments, the mixtures may comprise a buffer. In embodiments, such mixtures may be provided as substantially aqueous mixtures. In embodiments, the mixture may comprise a mixed aqueous/organic solvent system.

In embodiments, there are provided kits that comprise a tyrosinase enzyme and a functionalized TCO, along with instructions to conjugate the functionalized TCO to a biomolecule.

### IV. METHODS OF CONJUGATION

In embodiments, there are provided methods comprising adding a functionalized trans-cycloöctene (TCO) to an ortho-quinone present in a biomolecule, thereby forming a cycloadduct between the functionalized TCO and the ortho-quinone.

In embodiments, there are provided methods comprising providing a functionalized trans-cycloöctene (TCO), adding a protein or peptide comprising a phenolic moiety to the functionalized TCO, and generating an ortho-quinone from the phenolic moiety, wherein the functionalized TCO is allowed to react with the ortho-quinone to form a cycloadduct.

In embodiments, methods include generating the ortho-quinone by action of a tyrosinase enzyme. In embodiments, methods may comprise generating the o-quinone via chemical oxidation.

As described herein below in the Examples, TCO has surprisingly been shown to be the most efficient trap of transient o-quinones, which are prone to dimerization in the absence of a dienophile trap. In embodiments, the functionalized TCO is used in at least about a 5-fold molar excess relative to the protein or peptide. In embodiments, the functionalized TCO is used in at least about a 10-fold molar excess relative to the protein or peptide. In embodiments, the functionalized TCO is used in a 10- to 50-fold molar excess relative to the protein or peptide. In embodiments, the functionalized TCO is used in a 5- to 10-fold molar excess relative to the protein or peptide. In embodiments, the functionalized TCO is used in about a 5-fold molar excess relative to the protein or peptide

Any known linker strategy may be employed with the functionalized TCO to carry the cargo of the functionalized TCO. There are numerous commercially available products that have built in linkers on TCO, especially with polyethylene glycol (PEG) linkers. In embodiments, the methods may be carried out using a functionalized TCO comprising a TCO-PEG-acid, the acid linking to any desired R moiety as described herein.

In embodiments, the methods may be carried out using a functionalized TCO comprising a TCO-alcohol, the alcohol linking to any desired R moiety as described herein.

In embodiments, the methods may be carried out using a functionalized TCO comprising a TCO-PEG-amine, the amine linking to any desired R moiety as described herein.

In embodiments, the methods may be carried out using a functionalized TCO comprising a TCO-thiol or TCO-PEG-thiol.

In embodiments, the methods may be carried out using a functionalized TCO comprising a TCO-PEG-maleimide.

In embodiments, the methods may be carried out using a functionalized TCO comprising a TCO-PEG-OH.

In embodiments, the methods may be carried out using an ortho-quinone derived from a tyrosine moiety, the o-quinone being generated by action of a tyrosinase enzyme.

In embodiments, the methods may be carried out with a tyrosine at the C-terminal end of the protein.

In embodiments, the methods may be carried out with a tyrosine at the N-terminal end of the protein.

In embodiments, the tyrosine is located at an accessible internal location in the protein sequence. In embodiments, further amino acids can be incorporated to provide accessibility to the tyrosine residue by the tyrosinase.

Regardless of the position of tyrosine within the protein, techniques are available for site-selective incorporation. In embodiments, methods may further comprise engineering a protein with a site-specific tyrosine residue. In embodiments, the engineering step employs site-directed mutagenesis. In embodiments, semi-synthetic methods may be employed to incorporate tyrosine into a protein, including antibodies. Other techniques as described herein may also be used.

In embodiments, the methods may couple a protein that is an antibody fragment.

In embodiments, the methods may couple a protein that is a single-domain antibody.

### V. EXAMPLES

### General Procedures

All starting materials and solvents were obtained either from commercial sources or prepared according to the literature citation. Distilled water (in-house facility generation) was deionized (18 MQ). Tyrosinase from mushroom (T3824) was purchased from Sigma-Aldrich and used as received. DBCO-PEG₄-acid (BP-23760), endo-BCN-PEG₈-acid (BP-23768), and TCO-PEG₃-acid (BP-22420) were purchased from Broadpharm and used as received. Antibody fragments were generated in E. coli using a previously described expression and purification procedure. Tesar, et al. “Protein engineering to increase the potential of a therapeutic antibody Fab for long-acting delivery to the eye.” MAbs 2017, 9 (8), 1297-1305.

### Example 1

This Example shows the treatment of a human IgG1 Fab containing an engineered tyrosine with tyrosinase in pH 6 buffer at room temperatures resulting in rapid, transient observation of a 14 Da mass shift indicating presence of ortho-quinone.

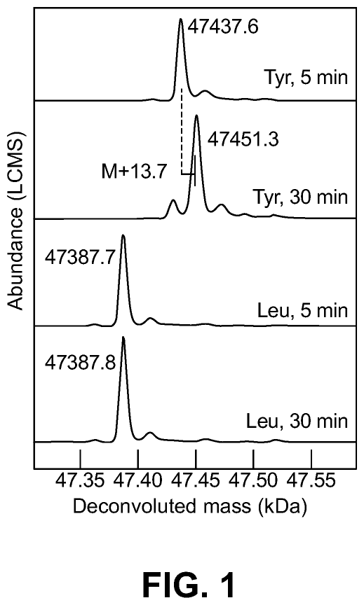

It has been indicated that human IgG1 framework antibodies do not contain tyrosine residues sufficiently exposed to react with tyrosinase. The present Example employed an antibody fragment (Fab) as a test substrate for bioconjugation. The mechanism of tyrosinase-mediated o-quinone generation in the absence of any partner reagent was first investigated. Incubation of a Fab containing tyrosine adjacent to C220 (EU numbering) at 1 mg/mL with 5% mol⁻¹ of mushroom tyrosinase at pH 6, resulted in a mass shift of the Fab by 14 Da as indicated in FIG. 1, and the appearance of a prominent UV band at 480 nm, which were assigned to o-quinone. The reaction was rapid with greater than 80% conversion in 30 minutes. A control substrate containing a leucine in place of tyrosine does not result in any change in mass or UV spectrum under the same conditions. The formation of dopaquinone was found to be efficient from pH 5-7.5, as measured by changes in mass and UV spectra, which is consistent with previous reports of tyrosinase activity.

The formation of o-quinone product is in competition with two notable side reactions. First, it was found that dopaquinone-containing Fabs form covalent dimers. The mass of the dimer is consistent with coupling of two o-quinone-containing Fabs, suggesting the reaction depends on lingering unreacted quinone. In addition, the UV spectrum of the dimer is blue shifted (to 410-420 nm) from the transient dopaquinone (480-500 nm), suggesting consumption of the quinone toward a more alkyl-like conjugate.

Second, fragmentation of the Fab to a lower molecular weight was detected. The mass of the fragment is consistent with cleavage on the amide backbone on the N-terminal side of the Tyr site. The fragmentation was faster at higher pH, suggesting a deprotonation followed by rearrangement to eliminate a new C-terminus. As indicated in the further Examples below, both reactions can be avoided by including a partner reagent to outcompete side product formation.

### Example 2

This Example shows the trapping of transient o-quinones with strained dieonophiles, in accordance with embodiments herein.

Standard reaction conditions consisted of 50-100 μM tyrosyl-Fab, 3-10 mol. equiv. of dienophile mol⁻¹ of tyrosine, pH 6, and 1% mol⁻¹ tyrosinase. A specific example is as follows: to a solution of Fab-GGY (5 mg, 1.14 mL at 4.4 mg/mL in sodium acetate, 20 mM, pH 5.0) in a 1.5 mL conical centrifuge tube were added buffer (MES, pH 6.0, 133 μL at 0.5 M), water (7 μL), TCO-PEG₄-acid (10.6 μL at 50 mM in water), and tyrosinase (46.2 μL at 5 mg/mL), resulting in a reaction composition of Fab (3.75 mg/mL, 79.7 μM), 5 mol. equiv. TCO (398 μM), buffer (50 mM), and tyrosinase (3.98 μM, 1% mol⁻¹). The tube was sealed and incubated at 25° C. while vortexing at 500 RPM. After 1.5 hours, the solution was purified by KappaSelect chromatography following the manufacturer's protocol, concentrated to 1.0 mL, and dialyzed against PBS, pH 7.4 (200 vol. equiv.). Fab [M]: starting material 47066.7 (calc'd); 47066.9 (found). Product: 47498.18 (calc'd); found 47498.24.

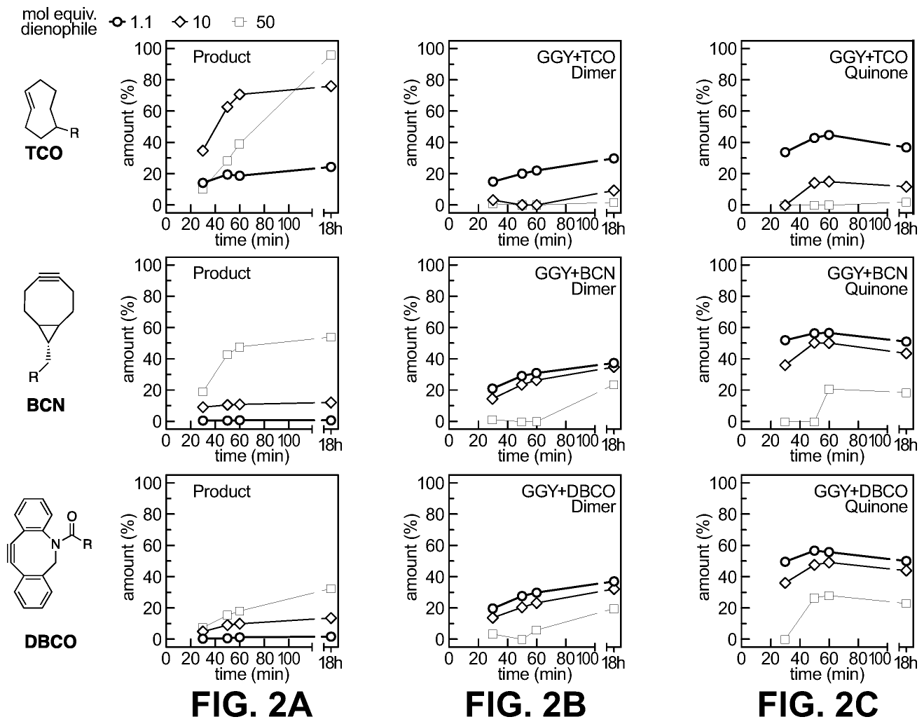

Reactions under the above condition were carried out with transcyclooctene (TCO), bicyclononane (BCN), and dibenzocyclooctyne (DBCO) as dienophile traps. Product formation time-course of TCO, BCN, and DBCO reagents toward dopaquinone generated in situ are shown in FIG. 2. As indicated in FIG. 2, the TCO based reagent was found to be a superior dienophile to trap the o-quinone. Even at high concentrations of the alternative dienophile reagents, the yields of product did not match those of the TCO based reagent. Moreover, BCN and DBCO reagents resulted in a large portion of unreacted o-quione (column FIG. 2B), which lead to formation of Fab dimer (column FIG. 2C); only TCO at 50 mol equivalents reacted sufficiently rapidly to avoid this byproduct. For dienophiles that react insufficiently fast with the transiently generated o-quinone, there is a significant portion of dimer or unreacted quinone in addition to the desired product.

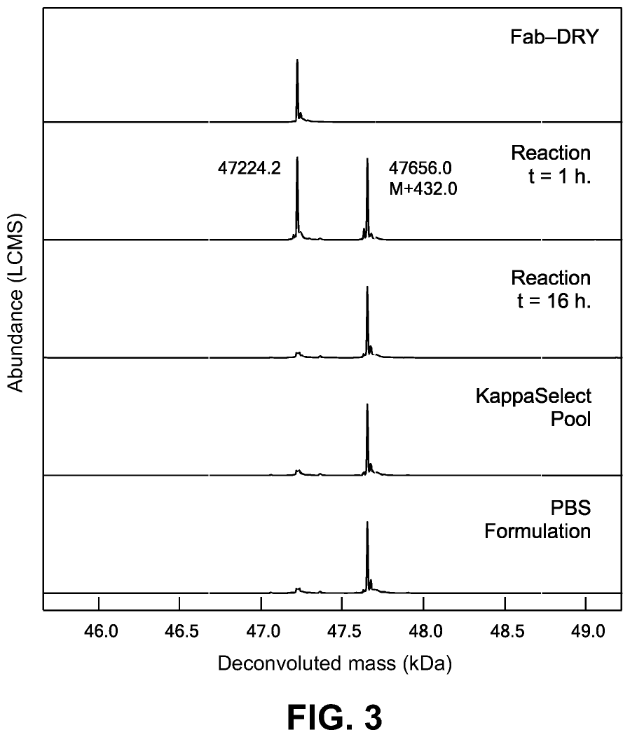

The spectra from top to bottom of FIG. 3 show starting material (Fab containing a C-terminal “DRY” peptide tag), the reaction mixture after one hour of reaction time showing the formation of conjugated product at M+432 Da (corresponding to 14 Da for +O, −H₂ and 428 Da for the TCO-PEG₄-COOH reagent), the reaction mixture after 16 hour of reaction time demonstrating about 91% yield, the purified pool after elution at pH 2.7 from a KappaSelect affinity column, and the final product formulated in PBS.

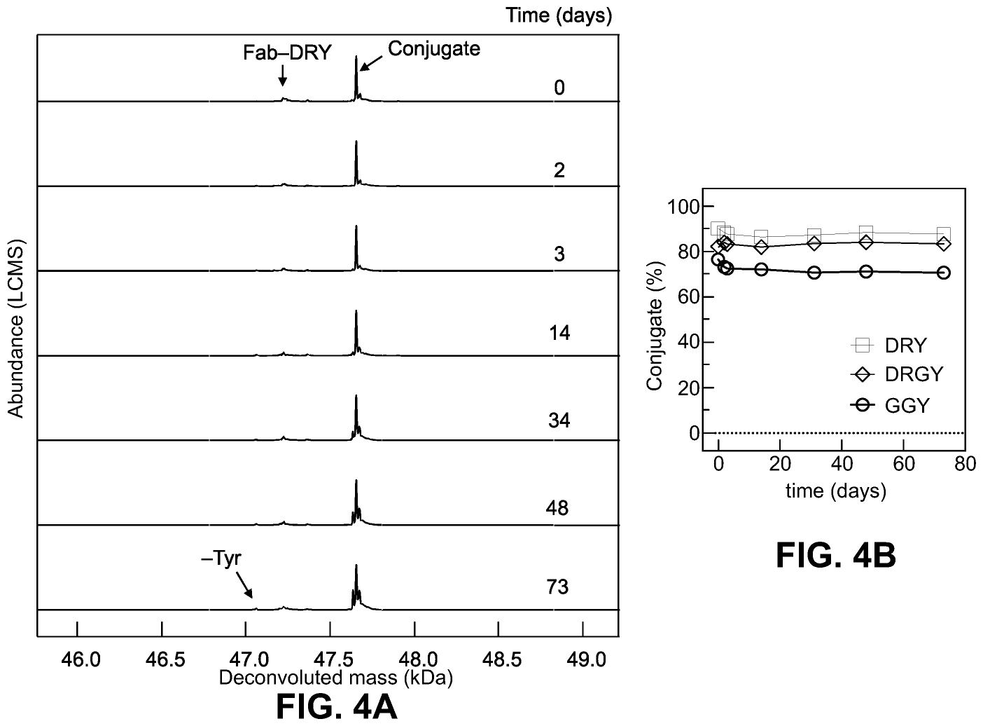

After affinity chromatography, each conjugate was formulated and stressed in a simple aqueous system (PBS pH 7.4, 37° C.). Stability was determined by LCMS monitoring of deconjugation, following free Fab and any released small molecule(s). Conjugates from TCO reagents appeared to be indefinitely stable under these mild conditions. No subsequent modification or deconjugation of TCO-based conjugates were observed at any timepoints up to 90 days. FIGS. 4A-B show stability of Diels-Alder cycloadducts, formed by tyrosinase-mediated bioconjugation, against PBS, pH 7.4, 37° C. over the course of multiple months for three variants of a Fab displaying an engineered C-terminal peptide tag: DRY, DRGY, and GGY. In each case, conjugates consisted of a mass shift corresponding to O-atom addition (16 Da), loss of H₂ (−2 Da), and addition of the mass of the TCO dienophile (TCO-PEG₄-carboxylic acid, 417.5); calculated total mass shift 431.5 Da. For Fab-GGY, found M+431.5; for Fab-DRY, found M+431.6; for Fab-DRGY, found 431.7. Each Fab-TCO-PEG₄-COOH conjugate was formulated at 5 mg/mL in PBS and sterilized in a tissue culture hood by passing through a 0.22 μm syringe filter. The container was sealed under ambient conditions, and then stored at 37° C. At each specified timepoint, an aliquot of sample was removed from the container and analyzed by LCMS for deconjugation. The amount of conjugate remaining was calculated as a percent of deconvoluted mass peak abundances. The left pane (FIG. 4A) shows the LCMS spectra recorded for the Fab-DRY protein. Peak abundance at the conjugate MW and the Fab starting material MW were largely unchanged, but there was an about 1% increase in abundance of a Fab fragment corresponding to loss of the C-terminal Tyr residue (calc'd M−163.5, found M−163.6). The right pane (FIG. 4B) is a summary of the amount of conjugate remaining at each timepoint for the three engineered, conjugated Fabs.

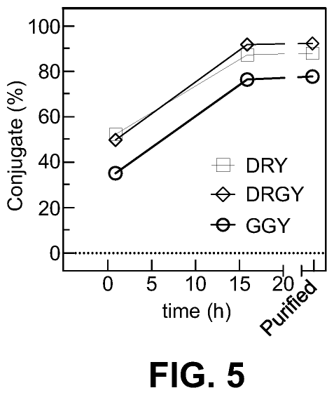

FIG. 5 shows a timecourse of reagent generation based on FIG. 4: proximal Arg leads to faster initial rate (1 hour) and higher overall yield. This provides an increase from 77% to 92.5% conjugation efficiency.

Conjugations were run at larger scale, and good performance was observed at 1 mol % tyrosinase, 5 mol equiv TCO, 80 uM tyrosine (Fab) reaction conditions. Surprisingly, the tag with an adjacent (DRY) or close (DRGY) arginine provide higher conjugation efficiency. Without being bound by theory, it is possible the Arg is activating/stabilizing the transient ortho-quinone, leading to higher yield. Side-by-side t=1 h and t=16 h timepoints for each of GGY, DRGY and DRY indicate the rate/yield trend holds. Accordingly, in embodiments, any protein (antibody of otherwise) may incorporate arginine adjacent or in proximity to a tyrosine residue to improve yields.

Although the foregoing invention has been described in some detail by way of illustration and Examples for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A composition comprising a cycloadduct of a functionalized trans-cycloöctene (TCO) with an ortho-quinone, wherein the ortho-quinone is present in a biomolecule.

2. The composition of claim 1, wherein the biomolecule is a protein or peptide.

3. The composition of claim 1, wherein the biomolecule is an oligosaccharide.

4. The composition of claim 1, wherein the biomolecule is a modified DNA or RNA.

5. The composition of claim 1, wherein the ortho-quinone is derived from a phenol-containing moiety.

6. The composition of claim 5, wherein the phenol-containing moiety is tyrosine.

7. The composition of claim 6, wherein the tyrosine is site-specifically engineered into a protein.

8. The composition of claim 5, wherein the phenol-containing moiety is a catechol.

9. The composition of claim 5, wherein when the biomolecule is an oligosaccharide, the oligosaccharide has a phenol-containing moiety at its reducing end.

10. The composition of claim 1, wherein the functionalized TCO comprises a protein, a second peptide, a drug, a nucleic acid, an oligosaccharide, a polymer, an oligomer, a dendrimer, a label, a diagnostic agent, a dual-function therapeutic-diagnostic agent, or a substrate surface, any one or more of which are optionally attached through a linker, wherein the linker is optionally branched.

11. The composition of claim 10, wherein the linker is branched and carries two or more of a protein, a peptide, a drug, a nucleic acid, an oligosaccharide, a polymer, an oligomer, a dendrimer, a label, a diagnostic agent, a dual-function therapeutic-diagnostic agent, or a substrate surface.

12. The composition of claim 10, wherein the nucleic acid is a RNAi or an anti-sense oligonucleotide.

13. The composition of claim 10, wherein the label is a fluorophore, a radiolabel, a chemiluminescent label, a DNA barcode, a RNA barcode, or a peptide tag.

14. The composition of claim 10, wherein the substrate surface is a polymer bead, a well bottom of a well-plate, or a polymer slide surface.

15. A composition of formula (I): 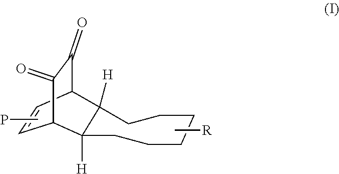
wherein P is a protein or peptide; and
R comprises a second protein, a second peptide, a drug, a nucleic acid, an oligosaccharide, a polymer, an oligomer, a dendrimer, a label, or a substrate surface, any of which are optionally attached through a linker.

16. The composition of claim 15, wherein P is an antibody, an antibody fragment, an enzyme, a cell surface protein, a cytokine, a chemokine, a protein toxin, or a hormone.

17-23. (canceled)

24. The composition of claim 15, wherein R comprises an antibody, a targeting molecule, a therapeutic agent, a radiolabel, a fluorescent label, a phosphorescent label, a dye, a polymer bead, a sensor surface, or a linker.

25-33. (canceled)

34. The composition of claim 15, where the composition has a structure of formula (II): 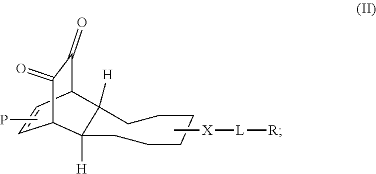
wherein L is a linker or bond and X is O, S, or NH.

35. The composition of claim 15, where the composition has a structure of formula (III): 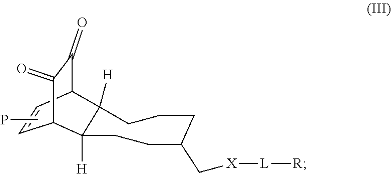
wherein L is a linker or bond and X is O, S, or NH.

36. A method comprising:
adding a functionalized trans-cycloöctene (TCO) to an ortho-quinone present in a biomolecule, thereby forming a cycloadduct between the functionalized TCO and the ortho-quinone.

37. A method comprising:
providing a functionalized trans-cycloöctene (TCO);
adding a protein or peptide comprising a phenolic moiety to the functionalized TCO; and
generating an ortho-quinone from the phenolic moiety, wherein the functionalized TCO is allowed to react with the ortho-quinone to form a cycloadduct.

38.-56. (canceled)

57. An antibody conjugate formed by action of tyrosinase on a phenolic residue in the antibody in the presence of a functionalized trans-cycloöctene (TCO), wherein the antibody conjugate is stable for at least one month at 37° C. in phosphate buffered saline.

58.-61. (canceled)

62. A protein conjugate formed by action of tyrosinase on a phenolic residue in the protein in the presence of a functionalized trans-cycloöctene (TCO), wherein the protein conjugate is stable for at least one month at 37° C. in phosphate buffered saline.

63.-67. (canceled)

68. A mixture comprising:
a biomolecule having a phenolic moiety;
a tyrosinase; and
a functionalized trans-cyclooctene.
